(19) United States
(12) Patent Application Publication
(10) Pub. No.: US 2010/0312149 A1
(43) Pub. Date: Dec. 9, 2010

(54) HARNESS SYSTEM FOR KINEMATIC ANALYSIS OF THE KNEE

(76) Inventors: Nicola Hagemeister, Montreal (CA); David Labbe, Granby (CA); Jacques De Guise, Montreal (CA); Rachid Aissaoui, Montreal (CA)

Correspondence Address: OGILVY RENAULT LLP, 1, Place Ville Marie, SUITE 2500, MONTREAL, QC, H3B 1R1, CA

(21) Appl. No.: 12/744,713

(22) PCT Filed: Nov. 26, 2008
(86) PCT No.: PCT/CA08/01509

§ 371 Date: Aug. 25, 2010

Related U.S. Application Data
(60) Provisional application No. 60/990,074, filed on Nov. 26, 2007.

(51) Int. Cl. A61B 5/103

(52) U.S. Cl. 600/595

(57) ABSTRACT

A harness for attachment about a knee femur of a subject, said harness comprising two abutment members, said abutment members being oriented against a skin outer surface at predetermined medial and lateral sites relative to a femur so as not to limit motion of the knee, and a strap operatively interconnecting the two abutment members such that the harness is adapted to be used on different knee sizes with the strap surrounding the knee and with the abutment members being urged against the skin outer surface at the predetermined medial and lateral sites by the strap, at least one of the abutment members supporting at least one femoral trackable member adapted to be tracked. The harness may be used for the pivot shift test.

### CROSS-REFERENCE TO RELATED APPLICATION

This application claims priority on U.S. Provisional Patent Application No. 60/990,074, filed on Nov. 26, 2007.

### FIELD OF THE APPLICATION

The present application relates to a knee harness and method for the precise and non-invasive measurement of knee motion and its analysis in 3D. Specifically, the present application measures precisely and non-invasively the relative 3D position and orientation, velocity and acceleration of the tibia in respect with the 3D position and orientation, velocity and acceleration of the femur during time and the relative 3D movement, velocity and acceleration of the tibia in respect of the femur.

### BACKGROUND OF THE ART

Human joints are usually more complex than a single axis. The knee joint is among the most complicated synovial joints in the musculoskeletal system. The kinematic studies of the knee allow the computation of relative movement during physical activities (such as walking), evaluating surgical operations such as ligament reconstruction, evaluating the effects of inaccurate positioning of condylar prostheses, evaluating the effect on the knee of the use of foot prostheses, evaluating diagnostic methods for ligament injuries and studying the injury mechanism in a knee joint.

By performing a combination of rolling and sliding, the knee joint accommodates the small contact area between the femur and the tibia. The anatomical structure of the femoral condyles leads to a complex combination of translations and rotations, which includes components of abduction/adduction, internal/external rotations and flexion/extension.

One test used to obtain such information is known as the pivot shift test. The pivot shift test is used for dynamically assessing the instability of the deficient knee following anterior cruciate ligament (ACL) injuries for example. The pivot shift test involves the patient lying down in supination, while a clinician performs movements and applies forces on the knee. With these manipulations, the clinician subjectively establishes the degree of instability of the knee. Due to the absence of non-invasive in vivo knee systems enabling the capture of objective data, the results of the pivot shift test as assessed by the clinician remain highly subjective.

The pivot shift is a complex, dynamic displacement between the tibia and the femur and no measurement tool or technique are currently commercially available to objectively assess the pivot shift. Rather, the clinician must subjectively attribute a grade of 0 (none), 1 (glide), 2 (clunk) or 3 (gross) to the shift on the basis of her/his experience. It is this grade which gives an appreciation of knee function. However, it has been well documented that different clinicians, especially less experienced ones, attribute grades differently for a same knee

In order to produce objective data during the pivot shift test, Hoshino et al. (“In Vivo Measurement of the Pivot-Shift Test in the Anterior Cruciate Ligament-Deficient Knee Using an Electromagnetic Device”, AJSM Preview, Mar. 9, 2007, doi: 10.1177/0363546507299447) describe the use of motion sensors on the leg to capture motion signals by which the movement of the leg is quantified. However, perhaps due to the nature of the motion sensors used and the fixation of these sensors on a simple elastic strap, or to an absence of normalization for the subjective assessment of the clinician, the results lacked accuracy.

U.S. Pat. No. 7,291,119, issued to De Guise et al. on Nov. 6, 2007, describes a harness that is used in 3D kinematic analysis of the knee. The harness is secured to predetermined sites on the knee, at which sites there is relatively little movement between the skin/soft tissue and the bone elements, whereby the harness is used non-invasively. The harness has a pair of abutment members that are interrelated by a rigid arch, with the arch supporting a trackable member. The harness does not impede the normal movement of the knee. One of the issues with the harness is that with its construction, it cannot be used with a patient in supination as the harness is maintained in position by abutment on a femoral condyle.

### SUMMARY OF THE APPLICATION

It is therefore an aim of the present application to provide a novel harness for kinematic analysis of the knee.

It is a further aim of the present application that the harness be used in the pivot shift test.

It is a still further aim of the present application to provide a method for normalizing the subjective results of the pivot shift test, for instance using the harness of the present application.

Therefore, in accordance with the present application, there is provided a harness for attachment about a knee of a subject, said harness comprising two abutment members, said abutment members being oriented against a skin outer surface at predetermined medial and lateral sites relative to a femur so as not to limit motion of the knee, and a strap operatively interconnecting the two abutment members such that the harness is adapted to be used on different knee sizes with the strap surrounding the knee and with the abutment members being urged against the skin outer surface at the predetermined medial and lateral sites by the strap, at least one of the abutment members supporting at least one femoral trackable member adapted to be tracked.

Further in accordance with the present application, each abutment member has at least one plate and an abutment projecting from the plate, the abutment made of a rigid material and being adapted to contact the skin outer surface at the predetermined site, with the plate contacting the skin outer surface in the periphery of the predetermined site.

Still further in accordance with the present application, each abutment member comprises two of said plate, with a first plate supporting the abutment, and a second plate connected to the first plate to define a gap therewith to accommodate the strap.

Still further in accordance with the present application, the plate supporting the abutment is sized 5 cm×6 cm.

Still further in accordance with the present application, the femoral trackable member is connected to a lateral one of the abutment members.

Still further in accordance with the present application, the femoral trackable member is at least one of a passive detectable device, an active detectable device, an accelerometer and a gyroscope.

Still further in accordance with the present application, the strap is made of an elastic material.

Still further in accordance with the present application, a first end of the strap has a loop surrounding the second plate of the medial abutment member so as to be connected thereto, with a second end of the strap passing through the gap of the lateral abutment member to be overlaid on the first end of the strap to surround the knee.

Still further in accordance with the present application, complementary Velcro™ strips are respectively provided on the first end and the second end of the strap to secure the ends of the strap to one another.

In accordance with another aspect of the present application, there is provided a harness system for tracking knee movements for a leg, comprising the harness as described above; and a tibial component comprising a support member adapted to be positioned against the skin on an anterior side of the tibia of the leg, the support member supporting at least one tibial trackable member adapted to be tracked, and a second strap for securing the tibial component to the tibia with the support member positioned against the skin on the anterior side of the tibia of the leg.

Still further in accordance with the present application, the support member comprises a rigid plate.

Still further in accordance with the present application, the second strap is connected at a first end to a first edge of the rigid plate, the second strap being looped through a channel at a second edge of the rigid plate to be overlaid on the first end of the strap to surround the tibia.

Still further in accordance with the present application, the tibial component further comprises a cushioning member on the rigid plate, the cushioning member oriented toward the tibia for interfacing the rigid plate to the tibia.

Still further in accordance with the present application, the rigid plate has a housing to accommodate the tibial trackable reference.

Still further in accordance with the present application, the tibial trackable member at least one of a passive detectable device, an active detectable device, an accelerometer and a gyroscope.

In accordance with yet another aspect of the present application, there is provided a harness for attachment about a knee of a subject, said harness comprising one abutment member, said abutment member being oriented against a skin outer surface at a predetermined lateral site relative to a femur so as not to limit motion of the knee, and a strap operatively interconnecting the abutment member such that the harness is adapted to be used on different knee sizes with the strap surrounding the knee and with the abutment member being urged against the skin outer surface at the predetermined lateral site by the strap, the abutment member supporting at least one femoral trackable member adapted to be tracked, the abutment member having at least one plate and an abutment projecting from the plate, the abutment made of a rigid material and being adapted to contact the skin outer surface at the predetermined site, with the plate contacting the skin outer surface in the periphery of the predetermined site.

In accordance with yet another aspect of the present application, there is provided a method for normalizing a pivot shift test on a knee, comprising tracking trackable members secured to the femur and to the tibia of a knee; measuring at least an orientation of the femur and of the tibia over time using tracking data from the trackable members during a pivot shift test; calculating a displacement of the femur with respect to the tibia and an angular velocity of flexion from the measured orientation; normalizing the measured displacement of the femur from a value related to said angular velocity of flexion of the knee; and grade the pivot shift test using the normalized displacement.

Further in accordance with the present application, calculating a displacement of the femur with respect to the tibia comprises calculating an acceleration of the knee, and normalizing the measured displacement of the femur comprises normalizing the measured displacement from a value related to said angular velocity of flexion of the knee and to said acceleration.

Still further in accordance with the present application, normalizing the measured displacement comprises calculating the value as:

log [(accel_{AP}+accel_{ML}+accel_{PD})/ω_{flexion}]

in which:

accel_{AP} is the anterior-posterior acceleration

accel_{ML} is the medio-lateral acceleration

accel_{PD} is the proximal-distal acceleration

ω_{flexion} is the mean angular velocity flexion.

### BRIEF DESCRIPTION OF THE DRAWINGS

FIG. 1A is a schematic view of a knee, from a medial standpoint;

FIG. 1B is a schematic view of the knee of FIG. 1A, from a lateral standpoint;

FIG. 2 is a perspective view of a harness constructed in accordance with an embodiment of the present application; and

FIG. 3 is a perspective view of a tibial component used in combination with the harness of FIG. 2.

### DESCRIPTION OF THE PREFERRED EMBODIMENTS

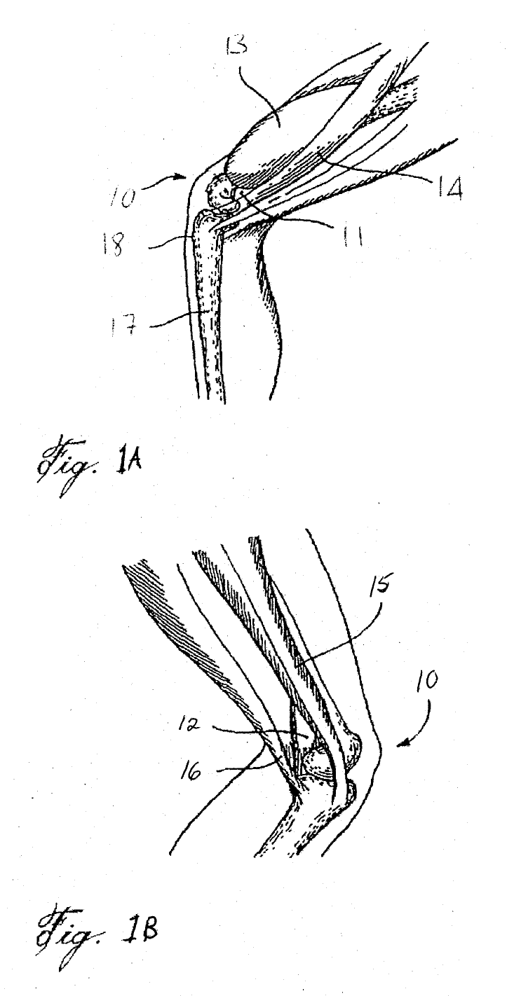

Referring to FIGS. 1A and 1B, parts of the knee 10 are described for reference, as the installation of the harness system of the present application will be related to the knee 10. The harness system features a harness that will be abutted against predetermined sites 11 and 12 on opposite sides of the knee. A first one of the predetermined sites 11 is located medially between the vastus medialis 13 and the sartorius tendon 14 of the knee 10. The predetermined site 12 is located laterally between the ilio-tibial band 15 and the biceps femoris tendon 16 of the knee 10. The sites 11 and 12 have been identified as locations on the knee 10 at which the relative movement between the skin/soft tissue and the bone elements is minimal, and negligible for the purposes of kinematic analysis.

The harness system of the present application also features a tibial component. Referring to FIGS. 1A and 1B, the position of the tibial component on the knee 10 is on the anterior side of the tibia 17, below the tuberosity 18.

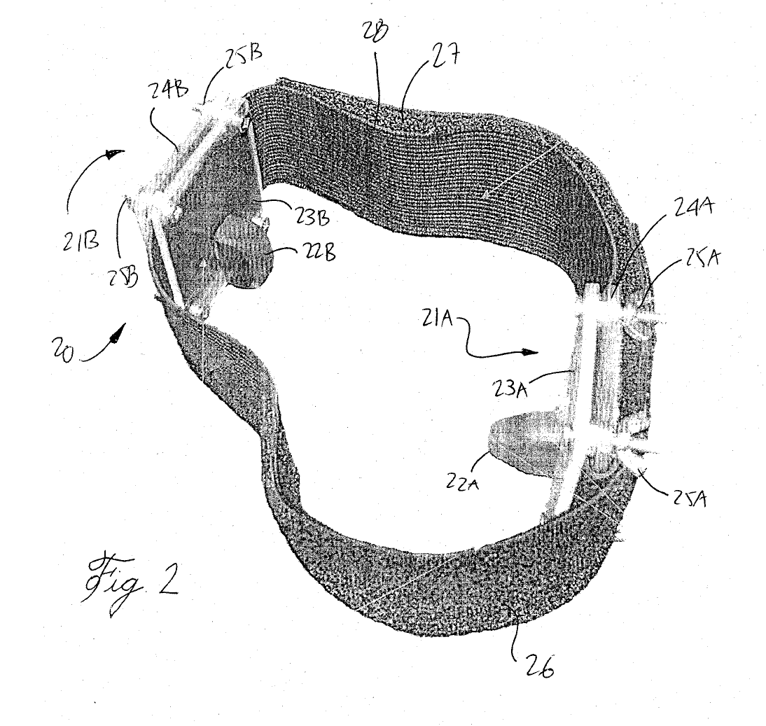

Referring to FIG. 2, the harness is generally shown at 20. The harness 20 is designed to be secured to the knee 10 (FIGS. 1A and 1B) at the predetermined sites 11 and 12, or other suitable locations on the knee 10.

The harness 20 has a pair of abutment members 21. The abutment members are illustrated as 21A and 21B, and their respective components will be appropriately affixed with “A” or “B” in the Figs.

Each abutment member 21 has an abutment 22 that will contact the knee 10 (FIG. 1) at the predetermined site 11 or 12. The abutments 22 are made of a material having a relatively high rigidity. For instance, the abutments 22 are made from a vulcanized rubber.

The abutments 22 each project normally from a support plate 23. The support plates 23 are made of a rigid material, such as polyvinyl chloride (PVC). Although there are numerous suitable dimensions considered for the support plates 23, a thickness of 0.3 cm for dimensions of 5 cm by 6 cm are suitable (i.e., approximately ⅛ in thickness, and 2×2⅜ inch). The dimensions of the support plates 23 are such that the support plates 23 contact the skin in the periphery of the predetermined sites 11 or 12. The combination of the abutments 22 and the support plates 23 contacting the knee ensures a suitable stability of each abutment member 21 with respect to the sits 11 and 12.

Each support plate 23 may be paired with a strap plate 24. The plates 24 are similar in construction to the plates 23. Each pair of support plate 23 and strap plate 24 defines a gap between plates. In the illustrated embodiment, the pairs of plates 23 and 24 are assembled to one another by fasteners 25 at the corners of the plates 23 and 24. Other configurations are also considered, as the fasteners 25 represent only solution amongst others. The fasteners 25 are typically nuts and bolts, with a spacer between the plates 23 and 24. In the illustrated embodiment, the fasteners 25 include a wing nut. This configuration is a possibility among numerous others to provide a gap between plates 23 and 24.

A strap 26 has an end looped about one of the abutment members 21 (i.e., abutment member 21A in FIG. 2). The other end of the strap 26 is therefore free and is passed through the gap of the other abutment member 21B, to then pass on an exterior of the abutment member 21A. The abutment member 21B is free to translate along the strap 26. The end of the strap 26 features a Velcro™ portion 27 so as to be secured to a complementing Velcro™ portion 28 elsewhere on the strap 26.

The strap 26 is made of a strip of material having a given level of elasticity, such as Neoprene. Accordingly, with the Velcro™ portions 27 and 28, and the translation between the strap 26 and the abutment member 21B, the harness 20 is securable to different knee sizes.

Either one of the abutment members 21 of the harness 20 supports one or more trackable members (not shown) for the 3D tracking of the harness 20, and thus of the femur. The femoral trackable member is any of active and passive trackable units, such as optical patterns of retro-reflective members or emitters (e.g., electromagnetic, RF, etc.). The trackable device is preferably positioned on the lateral one of the abutment members 21, namely the abutment member 21B. Other components may be provided on the abutment members 21, such as an accelerometer and a gyroscope.

It is pointed out that the oversizing of the strap plates 24 when compared to the abutments 22 reduces the area of contact between the knee 10 and the strap 26. The harness 20 may be provided with a single one of the abutment members 21. More specifically, in an embodiment, the harness 20 only features the lateral abutment member 21B.

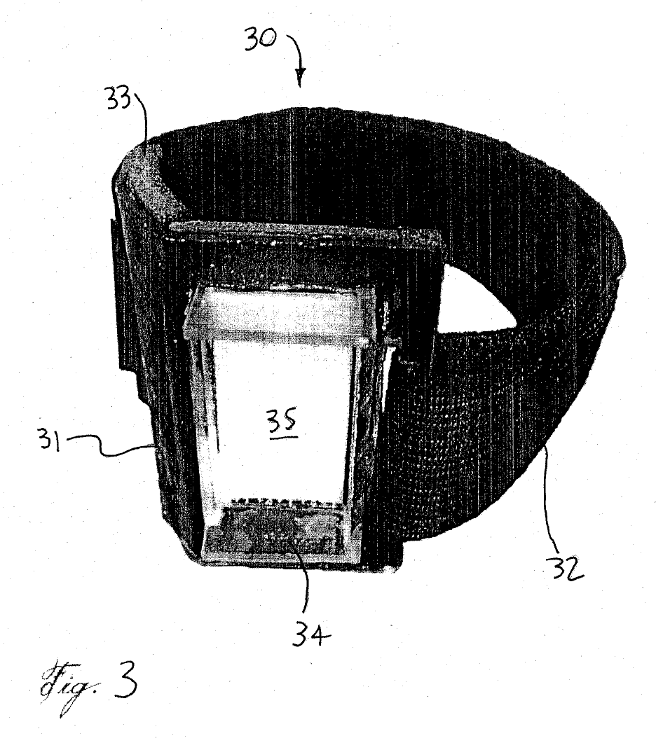

Referring now to FIG. 3, the tibial component is illustrated at 30. The tibial component 30 comprises a tibial support member 31 secured below the knee by means of an adjustable strap 32, or by other attachment means. As a non-restrictive example, the strap 32 has a width of 3.5 cm (i.e., approximately 1⅜ in). The strap 32 is preferably provided with appropriate Velcro™ strips to facilitate the installation of the tibial component 30 to the lower leg.

The support member 31 is curved in the shape of the tibia, to conform with the tibia when abutted against same. Cushioning member 33 is provided on the support member 31 to increase the comfort of the patient wearing the tibial component 30. A suitable thickness for the cushioning member 33 is 0.5 cm (approximately 3/16 in)), although other dimensions are considered. The support member 31 supports a trackable member housing 34 (similar to that used with the harness 20) The housing 34 is made of a rigid material (e.g., polyvinyl chloride), and accommodates one or more trackable members or sensors, such as electromagnetic position and orientation devices, accelerometers and gyroscopes. Suitable dimensions for the housing are 4 cm×2 cm, with a height of 1 cm (1½ in×¾ in×⅜ in), although other dimensions are considered. As illustrated, the housing 35 may incorporate a strip 35 of Velcro™ for quick connection of sensors/trackable members thereto. The tibial component 30 is configured and sized so as not to interfere with the clinician when the clinician applies forces on the knee 10 during the pivot shift test.

Briefly summarizing the method of determining the kinematic of a knee in a non-invasive manner comprising the harness system of the present application, the method comprises attaching the harness 20 about the knee 10. The tibial component 30 is then secured to the tibia so as to be substantially immovable with respect to the tibia.

Once the harness 20 and the tibial component are secured to the leg, data is generated by the tracking of the trackable members/sensors secured to the harness 20 and the tibial component 30 (in the trackable member housing 34). The data is treated, analyzed and resulting data is generated which describes the knee 10 to which the harness 20 and tibial component 30 are secured.

In installing the harness 20 about the knee 10 care is taken to place the abutments 21 in the predetermined sites 11 and 12 on the knee 10 (FIG. 1). The strap 26 is then manually tightened until the abutments 21 are fixed to the knee 10, while not impeding the normal movement of the knee 10. Once an appropriate tightness is reached, the harness 20 is secured using the Velcro™ portions 27 and 28.

The stability of the harness 20 is preferably verified after the knee 10 has been flexed a few times.

The tibial component 30 is installed by the support member 31 being positioned appropriately against the tibia, as discussed above. In the appropriate position, the strap 32 is tightened and secured to the tibia, without impeding the natural motion of the leg.

The analysis of data firstly involves defining a coordinate system relative to the trackable member fixed to the harness 20, and defining a coordinate system relative to the trackable member fixed to the tibial component 30. There are numerous prior-art ways to define such coordinate systems. For instance, one method is described in United States Publication No. 20050143676, published on Jun. 30, 2005 by De Guise et al. The coordinate systems are used to create three-dimensional representations of the femur and tibia and these representations accurately represent motions performed by the femur and tibia, relative to one another. Such tracking systems are well known. Additionally, electronic components such as accelerometers may be provided on the harness 20 and the tibial component 30. In the pivot shift test, the tibia and femur move relatively sharply with respect to one another. Accordingly, the use of an accelerometer may provide additional useful information.

The tracking of the harness 20 and of the tibial component 30 is performed when the knee 10 is in movement, for instance through the manipulations of the clinician in the pivot shift test.

Using the harness 20 and tibial component 30 or like harness system, the pivot shift test results may be normalized in accordance with the present application. In particular, the sum of linear accelerations (which is mainly composed of posterior and lateral accelerations) has a very strong correlation to the grade. The main component of the pivot shift is a posterior translation and is also generally coupled with an external rotation which has a component of lateral translation. The subjective grading system has an element of suddenness (clunk vs gross clunk) which can be characterized by acceleration.

As clinicians execute the pivot shift test with greater velocity of flexion generally produced kinematic parameters of greater amplitude, the normalization has all kinematic parameters related to the angular velocity of flexion produced by the clinician (e.g., mean angular velocity). For instance, the following normalization value is used for the knee:

log [(accel_{AP}+accel_{ML}+accel_{PD}/ω_{flexion}]

in which:

accel_{AP} is the anterior-posterior acceleration

accel_{ML}, is the medio-lateral acceleration

accel_{PD} is the proximal-distal acceleration

ω_{flexion} is the mean angular velocity of flexion

all of which take into account the measurements obtained from the combination of the harness 20 and the tibial component 30, or similar harness system. The value is then use to normalize the subjective results of grade from the clinician.

This normalised parameter shows differences between all pair of grades except between grades 0 and 1. A grade 1 represents a “glide” whereas a grade 0 is an absence of pivot shift. Therefore, they both present very small linear acceleration values and are better distinguished using the amplitude of posterior translation. Simple normalisation of kinematic data to account for the clinicians' different techniques allowed an improved correlation with the attributed grades.

1. A harness for attachment about a knee of a subject, said harness comprising two abutment members, said abutment members being oriented against a skin outer surface at predetermined medial and lateral sites relative to a femur so as not to limit motion of the knee, and a strap operatively interconnecting the two abutment members such that the harness is adapted to be used on different knee sizes with the strap surrounding the knee and with the abutment members being urged against the skin outer surface at the predetermined medial and lateral sites by the strap, at least one of the abutment members supporting at least one femoral trackable member adapted to be tracked.

2. The harness according to claim 1, wherein each abutment member has at least one plate and an abutment projecting from the plate, the abutment made of a rigid material and being adapted to contact the skin outer surface at the predetermined site, with the plate contacting the skin outer surface in the periphery of the predetermined site.

3. The harness according to claim 2, wherein each abutment member comprises two of said plate, with a first plate supporting the abutment, and a second plate connected to the first plate to define a gap therewith to accommodate the strap.

4. The harness according to claim 2, wherein the plate supporting the abutment is sized 5 cm×6 cm.

5. The harness according to claim 1, wherein the femoral trackable member is connected to a lateral one of the abutment members.

6. The harness according to claim 5, wherein the femoral trackable member is at least one of a passive detectable device, an active detectable device, an accelerometer and a gyroscope.

7. The harness according to claim 1, wherein the strap is made of an elastic material.

8. The harness according to claim 3, wherein a first end of the strap has a loop surrounding the second plate of the medial abutment member so as to be connected thereto, with a second end of the strap passing through the gap of the lateral abutment member to be overlaid on the first end of the strap to surround the knee.

9. The harness according to claim 8, wherein complementary Velcro™ strips are respectively provided on the first end and the second end of the strap to secure the ends of the strap to one another.

10. A harness system for tracking knee movements for a leg, comprising
a harness for attachment about a knee of a subject, said harness comprising two abutment members, said abutment members being oriented against a skin outer surface at predetermined medial and lateral sites relative to a femur so as not to limit motion of the knee, and a strap operatively interconnecting the two abutment members such that the harness is adapted to be used on different knee sizes with the strap surrounding the knee and with the abutment members being urged against the skin outer surface at the predetermined medial and lateral sites by the strap, at least one of the abutment members supporting at least one femoral trackable member adapted to be tracked; and
a tibial component comprising a support member adapted to be positioned against the skin on an anterior side of the tibia of the leg, the support member supporting at least one tibial trackable member adapted to be tracked, and a second strap for securing the tibial component to the tibia with the support member positioned against the skin on the anterior side of the tibia of the leg.

11. The harness system according to claim 10, wherein the support member comprises a rigid plate.

12. The harness system according to claim 11, wherein the second strap is connected at a first end to a first edge of the rigid plate, the second strap being looped through a channel at a second edge of the rigid plate to be overlaid on the first end of the strap to surround the tibia.

13. The harness system according to claim 11, wherein the tibial component further comprises a cushioning member on the rigid plate, the cushioning member oriented toward the tibia for interfacing the rigid plate to the tibia.

14. The harness system according to claim 11, wherein the rigid plate has a housing to accommodate the tibial trackable reference.

15. The harness system according to claim 10, wherein the tibial trackable member at least one of a passive detectable device, an active detectable device, an accelerometer and a gyroscope.

16. (canceled)

17. A method for normalizing a pivot shift test on a knee, comprising:
tracking trackable members secured to the femur and to the tibia of a knee;
measuring at least an orientation of the femur and of the tibia over time using tracking data from the trackable members during a pivot shift test;
calculating a displacement of the femur with respect to the tibia and an angular velocity of flexion from the measured orientation;
normalizing the measured displacement of the femur from a value related to said angular velocity of flexion of the knee; and
grade the pivot shift test using the normalized displacement.

18. The method according to claim 17, wherein calculating a displacement of the femur with respect to the tibia comprises calculating an acceleration of the knee, and normalizing the measured displacement of the femur comprises normalizing the measured displacement from a value related to said angular velocity of flexion of the knee and to said acceleration.

19. The method according to claim 18, wherein normalizing the measured displacement comprises calculating the value as:
log [accel_{AP}+accel_{ML}+accel_{PD})/ω_{flexion}]
in which:
accel_{AP} is the anterior-posterior acceleration
accel_{ML} is the medio-lateral acceleration
accel_{PD} is the proximal-distal acceleration
ω_{flexion} is the mean angular velocity of flexion.
